(19) United States
(12) Patent Application Publication
(10) Pub. No.: US 2016/0270763 A1
(43) Pub. Date: Sep. 22, 2016

(54) SYNTHETIC APERTURE ULTRASOUND SYSTEM

(71) Applicant: Decision Sciences Medical Company, LLC, Poway, CA (US)

(72) Inventors: James J. Hayes, Fort Irwin, CA (US); Evan Freiburg, San Diego, CA (US); Dustin Kruse, Grand Island, NY (US)

(21) Appl. No.: 15/072,324

(22) Filed: Mar. 16, 2016

Related U.S. Application Data
(60) Provisional application No. 62/135,066, filed on Mar. 18, 2015.

(51) Int. Cl. A61B 8/00; A61B 8/08; A61B 8/14

(52) U.S. Cl. CPC A61B 8/4494; A61B 8/145; A61B 8/54; A61B 8/0825; A61B 8/0808; A61B 8/4281; A61B 8/085; A61B 8/0875; A61B 8/5207; A61B 8/5223; A61B 8/4461

(57) ABSTRACT

Systems, devices, and methods for synthetic aperture acoustic imaging, range-Doppler measurements, and therapies are disclosed. One synthetic aperture acoustic system includes a waveform generation and processing device and an acoustic probe device that are designed to enable generation, transmission, reception, and processing of coherent, spread-spectrum, instantaneous-wideband, coded waveforms in synthetic aperture ultrasound (SAU) applications.

### CROSS REFERENCE TO RELATED APPLICATIONS

This patent document claims the benefit of priority of U.S. Provisional Patent Application No. 62/135,066, filed on Mar. 18, 2015. The entire content of the before-mentioned patent application is incorporated by reference as part of the disclosure of this document.

### TECHNICAL FIELD

This patent document relates to systems, devices, and processes for acoustic energy diagnostics and therapies.

### BACKGROUND

Acoustic imaging is an imaging modality that employs the properties of sound waves traveling through a medium to render a visual image. High frequency acoustic imaging has been used as an imaging modality for decades in a variety of biomedical fields to view internal structures and functions of animals and humans. High frequency acoustic waves used in biomedical imaging may operate in different frequencies, e.g., between 1 and 20 MHz, or even higher frequencies, and are often termed ultrasound waves. Some factors, including inadequate spatial resolution and tissue differentiation, can lead to less than desirable image quality using conventional techniques of ultrasound imaging, which can limit its use for many clinical indications or applications.

### SUMMARY

Systems, devices, and methods for synthetic aperture acoustic imaging, range-Doppler measurements, and therapies are disclosed. In one embodiment, a synthetic aperture acoustic waveform system includes a waveform generation and processing device and an acoustic probe device. The waveform generation and processing device includes a waveform generator in communication with one or more waveform synthesizers to generate one or more waveforms according to waveform information provided by the waveform generator, and a controller unit including a memory to store data and a processing unit coupled to the memory to process data. The acoustic probe device a housing body including a shaped section to interface a body structure of a biological subject, and one or more transducer segments comprising an array of transducer elements arranged on the shaped section of the housing body to transmit acoustic waveforms corresponding to the one or more waveforms generated by the waveform generation and processing device toward a target volume in the biological subject and to receive returned acoustic waveforms that return from at least part of the target volume. The acoustic probe device further includes an acoustic coupling component to conduct the acoustic waveforms between the transducer elements and the body structure of the biological subject when in contact with the acoustic coupling component, and a multiplexing unit in communication with the controller unit and the array of transducer elements to select one or more transducing elements of the array to transduce the waveforms into the corresponding acoustic waveforms, and to select one or more transducing elements of the array to receive the returned acoustic waveforms.

In the above noted embodiment, the waveform generation and processing device includes an array of analog to digital (A/D) converters to convert the received returned acoustic waveforms received by the array of transducer elements of the acoustic probe device from analog format to digital format as a received waveform that includes information of at least part of the target volume, and one or more amplifiers in communication with the one or more waveform synthesizers to modify the individual orthogonal coded waveforms provided to the acoustic probe device for transmission. The waveform generation and processing device further includes one or more pre-amplifiers in communication with the acoustic probe device and the array of A/D converters to modify the received returned acoustic waveforms provided to the A/D converters.

### BRIEF DESCRIPTION OF THE DRAWINGS

FIG. 1 shows a block diagram of one example embodiment of a synthetic aperture acoustic system of the disclosed technology.

FIG. 2A shows a block diagram of an example architecture of a 64-channel transmit/receive electronics module (TREM).

FIG. 2B shows an image of an example TREM unit.

FIG. 2C shows a diagram of an example backplane of the TREM unit on a printed circuit board.

FIG. 2D shows a data flow diagram of the example synthetic aperture acoustic system.

FIG. 3A shows a block diagram of an example control unit of the TREM unit.

FIG. 3B shows a block diagram of clocking using the control unit.

FIG. 3C shows an example printed circuit board design including the control unit of the TREM unit.

FIG. 4A shows a block diagram of an example circuit of a Tx/Rx card of the TREM unit.

FIG. 4B shows a block diagram of a 16-channel selection circuit of the Tx/Rx circuit.

FIG. 4C shows a block diagram of clocking operated via the Tx/Rx card.

FIG. 4D shows an example printed circuit board design of a Tx/Rx card of the TREM unit.

FIG. 5 shows an example printed circuit board design of a power amplifier of the TREM unit

FIG. 6 shows an example printed circuit board design of the probe interface adapter of the TREM unit.

FIG. 7A shows a breakout schematic diagram of an example acoustic probe device.

FIG. 7B shows a schematic illustration of an exemplary embodiment of the acoustic probe device.

FIG. 8 shows an example printed circuit board design for the transducer array L0 probe unit of the exemplary acoustic probe device.

FIG. 9 shows an example data flow diagram for probe data between the TREM unit and the exemplary probe device.

FIG. 10 shows a block diagram of the multiplexer units of the exemplary probe device.

FIG. 11 shows a block diagram of an example transducer element arrangement on transducer segments.

FIG. 12 shows a diagram of exemplary composite ultrasound beams generated by transducer sub-arrays on multiple transducer segments that forms a synthetic aperture beam from multiple transmitting positions along a 180° curvature of the acoustic probe.

### DETAILED DESCRIPTION

In this description, the word “exemplary” is used to mean serving as an example, instance, or illustration. Any embodiment or design described herein as “exemplary” is not necessarily to be construed as preferred or advantageous over other embodiments or designs. Rather, use of the word exemplary is intended to present concepts in a concrete manner.

Acoustic imaging can be performed by emitting an acoustic waveform (e.g., pulse) within a physical elastic medium, such as a biological medium, including tissue. The acoustic waveform is transmitted from a transducer element (e.g., of an array of transducer elements) toward a target volume of interest (VOI). Propagation of the acoustic waveform in the medium toward the target volume can encounter structures that cause the acoustic waveform to become partly reflected from a boundary between two mediums (e.g., differing biological tissue structures) and partially transmitted. The reflection of the transmitted acoustic waveform can depend on the acoustic impedance difference between the two mediums (e.g., at the interface between two different biological tissue types). For example, some of the acoustic energy of the transmitted acoustic waveform can be scattered back to the transducer at the interface to be received, and processed to extract information, while the remainder may travel on and to the next medium. In some instances, scattering of the reflection may occur as the result of two or more impedances contained in the reflective medium acting as a scattering center. Additionally, for example, the acoustic energy can be refracted, diffracted, delayed, and/or attenuated based on the properties of the medium and/or the nature of the acoustic wave.

Acoustic imaging system transducers may employ an array of piezoelectric elements to transmit an acoustic pulse toward the target VOI (e.g., target biological tissue) and receive the returned acoustic signals (echoes) that return from scattering structures within. In such systems, the transducer array functions as the aperture of the imaging system. The acoustic waveforms (e.g., ultrasound pulses) can be electronically steered and focused as a sequence pulses through a plane or volume and used to produce a 1D, 2D and/or 3D map of the returned echoes used to form an image of the target. Beamforming can occur both in transmit and receive. In transmit, for example, beamforming can include the utilization of phase differences between channels to form, focus and steer the beam. In some implementations, the ultrasound pulse and the returned echoes transmitted and received at the transducer array can be individually delayed in time at each transducer of the array to act as a phased array.

In conventional real aperture ultrasound imaging systems, the quality of images directly depends on the acoustic field generated by the transducer of the ultrasound system, and the image is typically acquired sequentially, one axial image line at a time (i.e., scan of the target area range slice by slice). This sets limits on the frame rate during imaging that may be detrimental in a variety of real-time ultrasound imaging applications, e.g., including the imaging of moving targets.

To address limitations with conventional real aperture ultrasound imaging, synthetic aperture ultrasound imaging can be used to improve the quality of ultrasound images. A “synthetic aperture” is the concept in which the successive use of one or more smaller, real apertures (sub-apertures) to examine a VOI, whose phase centers are moved along a known one-dimensional (1D), two-dimensional (2D), and/or three-dimensional (3D) path of a particular or arbitrary shape, is implemented to realize a larger effective (non-real) aperture for acquiring an image. The synthetic aperture can be formed by mechanically altering the spatial position of the electro-acoustic transducer (e.g., transducer array) to the successive beam transmission and/or receiving locations, by electronically altering the phase center of the successive beam transmission and/or receiving locations on the electro-acoustic transducer array, or by a combination of both. Synthetic aperture-based imaging was originally used in radar systems to image large areas on the ground from aircraft scanning the area of interest from above. Synthetic aperture focusing in ultrasound imaging is based on the geometric distance from the ultrasound transmitting elements to the VOI location and the distance from that location back to the ultrasound receiving element. In ultrasound imaging, the use of the synthetic aperture enables the focusing on a point in the target region by analyzing the received amplitude and phase data of the returned echoes (e.g., mono-static and bi-static echoes), recorded at each of a plurality of transmitter and receiver positions from all directions, to provide information about the entire area. Since the direction of the returned echoes cannot be determined from one receiver channel alone, many receiver channels are used to determine the information contained in the returning echoes, which are processed across some or all of the channels to ultimately render information used to produce the image of the target region.

Systems, devices, and methods for synthetic aperture acoustic imaging, range-Doppler measurements, and therapies are disclosed. In some implementations, the disclosed synthetic aperture acoustic systems include an architecture designed for generating, transmitting, receiving, and processing coherent, spread-spectrum, instantaneous-wideband, coded waveforms in synthetic aperture ultrasound (SAU) applications.

The disclosed SAU systems can provide high definition (HD) and enhanced image quality, contrast and resolution, e.g., as compared to existing ultrasound imaging techniques, and can enable tissue differentiation and classification of imaged structures in the target VOI. The disclosed SAU systems include specialized hardware designs to generate, transceive, and process multiple types of waveforms, e.g., including arbitrary linear or nonlinear waveforms, or coded waveforms. The arbitrary waveforms or the coded waveforms can be coherent, transceived instantaneously, and/or spread across a selected region or regions of a spectrum. A SAU system of the disclosed technology can generate individual arbitrary waveforms or the coded waveforms at selected transducer elements to form one or more composite waveforms that are each formed of two or more individual waveforms to be transmitted (as acoustic waveforms) toward a target VOI from one or multiple transmitting positions of a transducer array of the SAU system, and receive the returned acoustic waveforms from one or multiple receiving positions of the transducer array. In an example, a composite waveform of three individual arbitrary waveforms, some or all of which may be coded waveforms, are generated by the disclosed SAU system and transceived from transmitting/receiving positions at various selected transducer elements, in which the first individual arbitrary or coded waveform includes a first center frequency ω₁, the second individual arbitrary or coded waveform includes a second center frequency ω₂, and the third individual arbitrary or coded waveform includes a third center frequency ω₃, in which each individual waveform is generated with the same or differing amplitudes or phases.

For example, the system architecture provides the capability of generating any arbitrary waveform (e.g., described mathematically) to be beamformed, transmitted, and steered and/or focused. Similarly, for example, the system allows for the generation of composite waveforms that are formed of two or more individual coded waveforms to be transmitted toward and received from the target VOI. The individual coded waveforms of the composite waveform are mutually orthogonal to each other and are in different frequency bands, such that each of the individual mutually orthogonal coded waveforms includes a unique frequency with a corresponding phase. The transmitting and receiving positions of the transducer array can include selected spatial positions of the transducer array with respect to the target VOI for a composite waveform transmitted and received, respectively, from a selected transducer subarray of transducer elements of the transducer array. The transmitting and receiving positions of the transducer array can include selected beam phase center positions of the transducer array for a composite waveform transmitted and received, respectively, from a selected transducer subarray of the transducer array.

Additional information pertaining to the disclosed coherent, spread-spectrum, instantaneous-wideband, coded waveforms is described in the U.S. Pat. No. 8,939,909 and U.S. patent application Ser. No. 14/479,249, titled COHERENT SPREAD-SPECTRUM CODED WAVEFORMS IN SYNTHETIC APERTURE IMAGE FORMATION (published as U.S. patent application with Publication No. 2015/0080725), which are incorporated by reference as part of this disclosure in this patent document.

The use of coherent waveforms in implementations of the disclosed SAU systems can permit the complex correlation of a portion of, or the entire, echo return with a selected reference signal, such as, for example, the transmitted waveform. Such coherent complex correlations permit the reduction of image and signal artifacts and the extraction of data at lower signal-to-noise ratios and in the presence of interference.

The use of spread-spectrum signals in implementations of the disclosed SAU systems can allow the definitive design of acoustic waveforms that have deliberate and explicit amplitude and phase frequency content. For example, by explicitly defining the amplitude and/or phase of each frequency component of the spread-spectrum composite acoustic waveforms can be constructed such that signal and information processing techniques can be employed to extract the maximal amount of information from the echo returns, e.g., approaching mathematical limits.

The use of instantaneous coherent, wideband, spread-spectrum, coded waveforms in implementations of the disclosed SAU systems can enable the capture of all available information during each transmit-receive interval, e.g., thereby minimizing the corruption of the returned signal by the inhomogeneous, dynamic nature of living biological specimens, and by motion induced artifacts of the collection process. Additionally, for example, fundamental physical parameters (e.g., such as bulk modulus, density, attenuation, acoustic impedance, amplitude reflections, group delay, or other) can be extracted by using signal and information processing methods of the disclosed technology to enable differentiation and classification of the tissue in the VOI. For example, some signal and information processing methods of the disclosed SAU technology may include inverse mathematical techniques operating on the received frequency and angular dependent wideband, spread-spectrum, synthetic aperture received signal echoes for differentiating and/or classifying tissue in the VOI, as well as expert system techniques, e.g., deterministic, support vector network and neural network techniques.

Explicit amplitude and/or phase coding of each frequency component of waveforms in implementations of the disclosed SAU systems can provide multiple benefits. For example, amplitude coding allows for the explicit compensation of the frequency-dispersive properties of the transducer array and of the acoustic propagation channel. The amplitude and/or phase coding of each frequency component permits deterministic beamforming and steering of wide-instantaneous waveforms. Explicit amplitude and phase coding of each frequency component of an exemplary transmitted signal permits the minimization of the peak-to-average power ratio (PAPR), and the spreading of the acoustic power over a wide band, e.g., to minimize deleterious biological effects. For example, by explicitly defining the amplitude and/or phase of each frequency component of spread-spectrum signals, waveforms can be constructed that may be transmitted simultaneously, which exhibit minimal interference with each other, such that signal and information processing techniques can be employed to recover the received signal associated with each individual transmitted waveform. Further, the coded, spread-spectrum acoustic waveforms of the disclosed SAU technology can allow for motion compensation due to particular ambiguity properties of these waveforms.

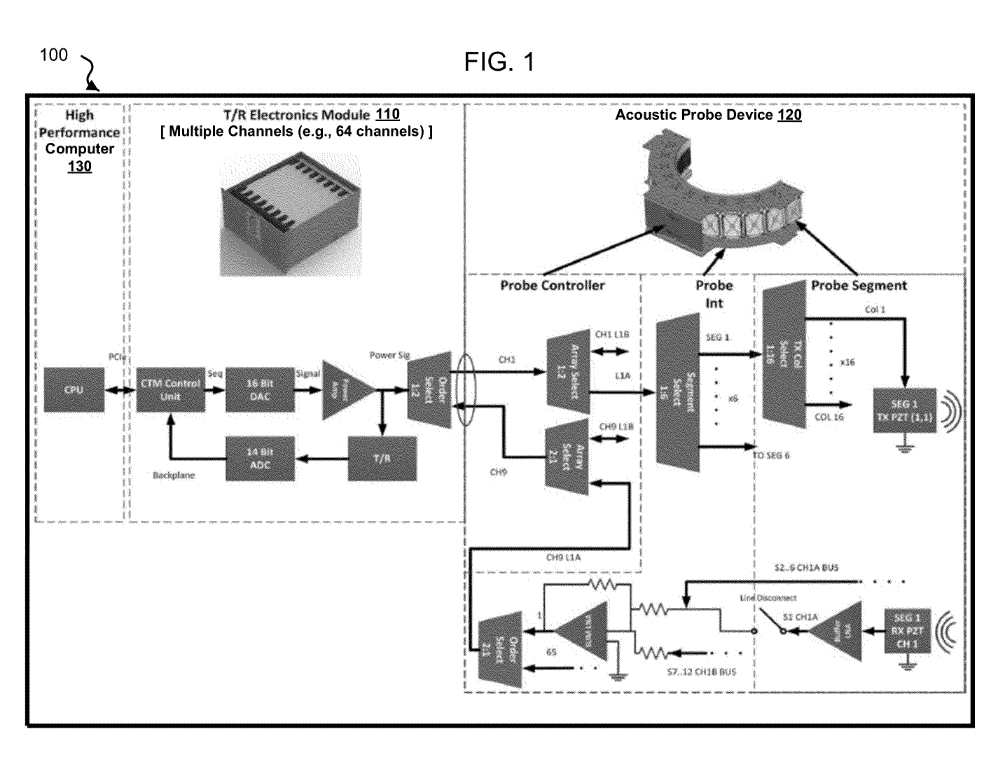

FIG. 1 shows a block diagram of one example embodiment of a synthetic aperture acoustic system 100 of the disclosed technology. As shown in FIG. 1, the SAU system 100 includes a transmit/receive electronics module (TREM) 110 in electrical communication with an acoustic probe device 120 and with a data processing unit or computer 130. The TREM 110 is configured to generate the individual coded waveforms on multiple channels transferred to the probe device 120 for transmitting and receiving one or more composite waveforms (e.g., coherent, spread-spectrum, instantaneous-wideband, coded waveforms) based on the individual-generated coded waveforms. The TREM 110 includes a waveform generator unit that includes a function generator and an arbitrary waveform generator (AWG). The TREM 110 includes a system control unit to control the waveform generator unit for the synthesis of individual coded waveforms. The TREM 110 includes signal conditioning and processing circuitry to amplify, select, and/or convert analog and digital signals, e.g., which can include analog/digital converters, multiplexers, amplifiers, etc. The TREM 110 includes a data processing unit (e.g., processor or microcontroller, and memory) is configured to transfer data with a central processing unit (CPU) of the computer 130, e.g., such as executable instructions on waveform synthesis or probe control, and/or acquired or processed data.

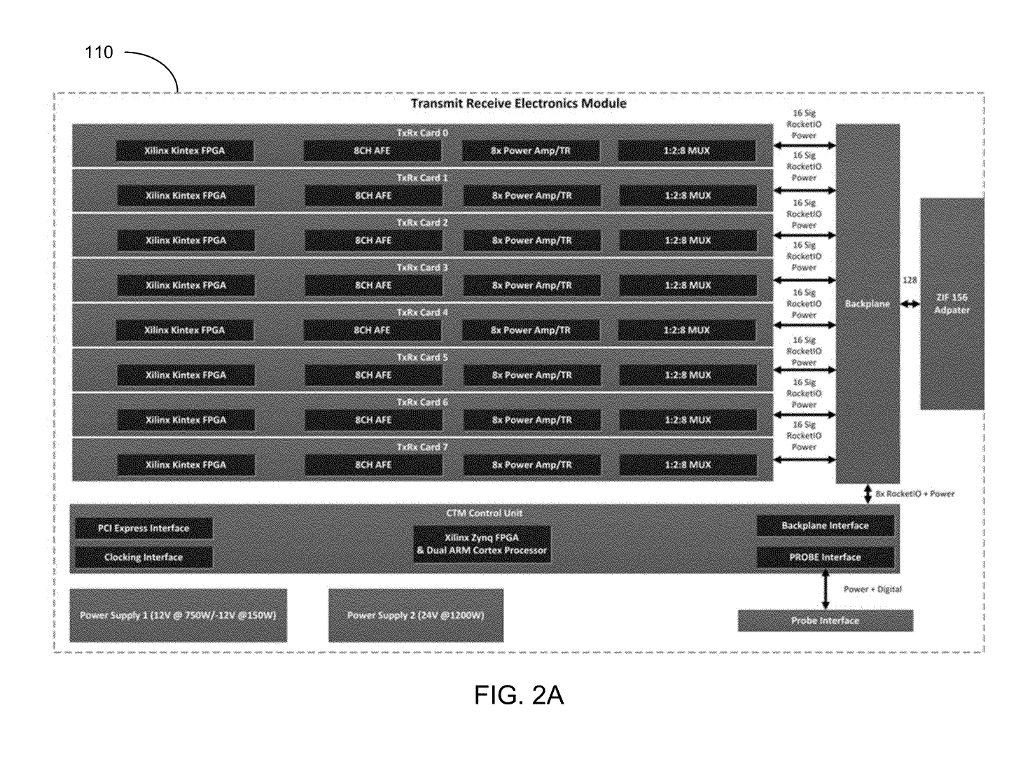

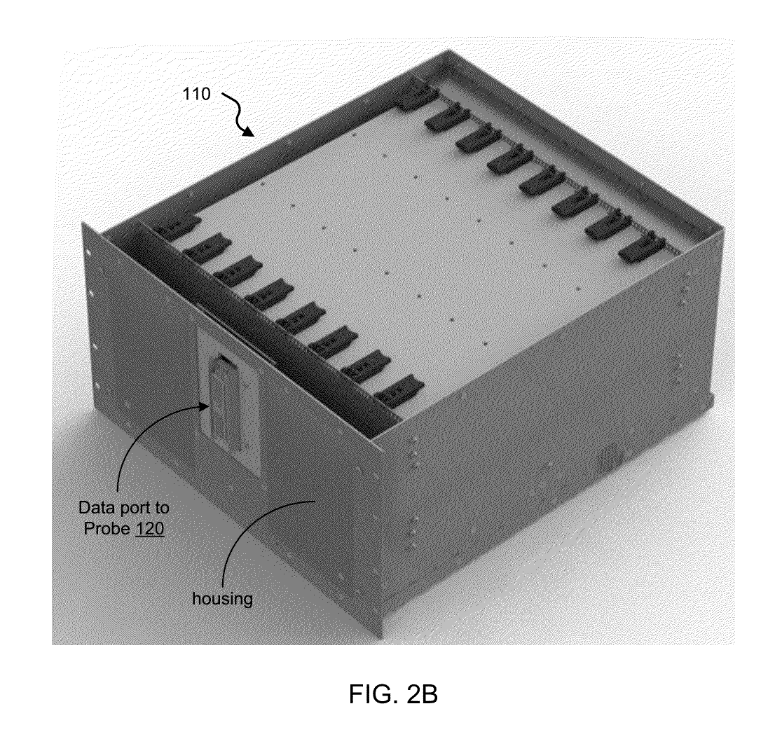

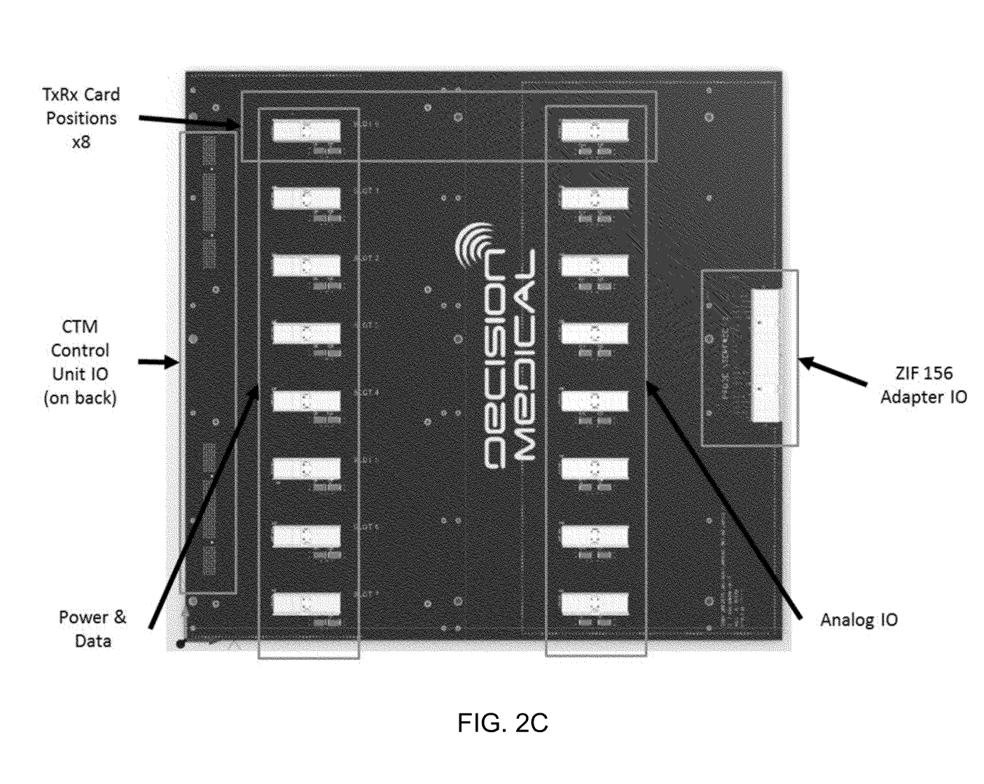

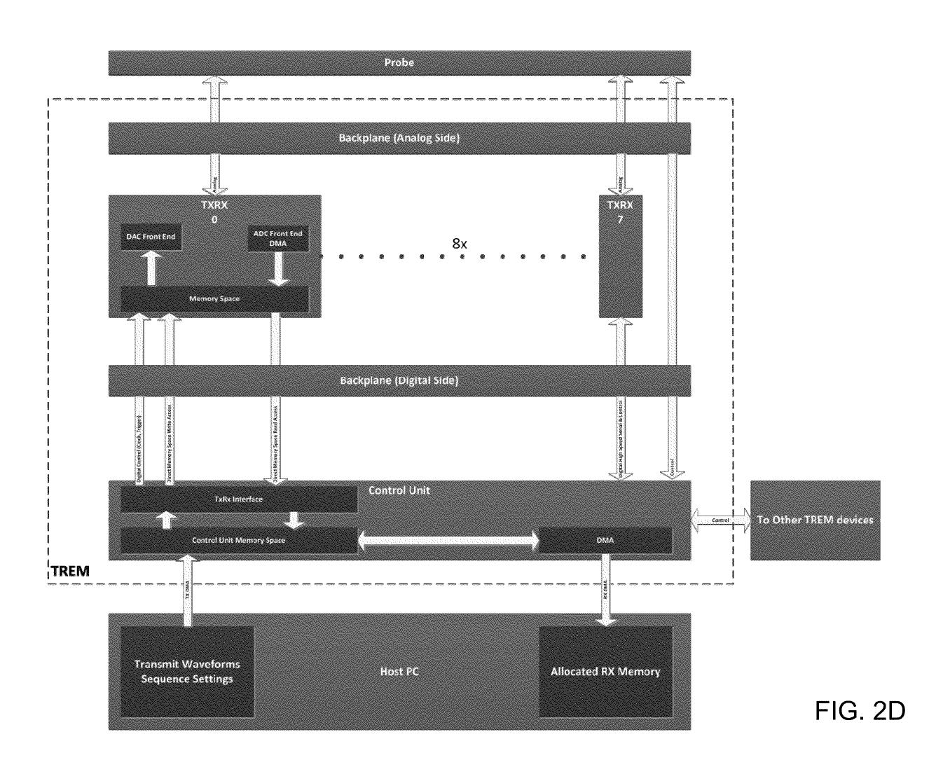

The TREM 110 is a highly parallelized, high bandwidth signal processing system that includes following subsystems. The TREM 110 includes function generators and/or arbitrary waveform generators (AWG) 110(a), which includes hardware digital-to-analog converter (DAC) and sequencing logic/memory/firmware. In the example shown in FIG. 2A, the TREM 110 is configured to include 64 channels, but can be configured to provide 128 channels. In other examples, waveform generators of the TREM 110 can provide greater than 128 channels, e.g., 256, 512, or more. The TREM 110 includes a System Sequencer 110(b), responsible for real time configuration of the other subsystems. The TREM 110 includes Channel Multiplexing circuitry 110(c), used to route the primary transmitter/receivers to multiple of individual elements, e.g., tens of thousands in theory. In one example, the TREM 110 is able to select between two arrays (e.g., a transmit array and a receive array, each organized in individual arrays on individual transducer segments), e.g., where one array includes 1536 transducer elements and the other array includes about 8000 transducer elements. Both arrays are capable of acting on the same or adjacent VOIs. The channel multiplexing circuitry can span between the TREM 110 and the acoustic probe device 120. The TREM 110 includes a Trigger and Clocking system 110(d), used to synchronize the internal subsystems with extremely precise/low jitter clock networks and trigger distribution. The clock and trigger originates in the CCU (e.g., also referred to as the Control Unit) or from an external device or system (when slaved) and is distributed over the backplane of the TREM 110. The TREM 110 includes Transmit Drivers 110(e), which include highly linear transformer coupled power amplifiers used to drive the piezoelectric elements. In some examples, the TREM 110 can include 64 transmit drivers, whereas in other examples, 128 transmit drivers. The TREM 110 includes Front End Receiver and ADC 110(f), which includes hardware analog-to-digital converter (ADC) and sequencing logic/memory/firmware and a DMA based data delivery chain. The TREM 110 includes Inter-device Communication Channels 110(g), which are part of the backplane of the system 100, e.g., which in some implementations be configured to utilize a standard serial signaling protocol called Aurora over a Xilinx serial physical layer that provides tremendous bandwidth between each transmitter card (Tx/Rx, 8 per CTM) and the system controller (CCU, 1 per CTM). In addition to the trigger and clock, the CCU accesses the memory space on each Tx/Rx card and coordinates DMA transfers from those devices to the host PC via an external PCI Express link. The TREM 110 includes Probe Control and data path 110(h), which can be used for probe channel selection/sequencing. Additionally, or alternatively, for example, the probe 120 can also include an FPGA on board used for channel selection/sequencing. However, this device is connected by a medium speed serial link and could include waveform generation and capture (ADC/DAC) locally on the probe head. This would eliminate much of the analog connections and circuitry used in the present design of the TREM 110, for example. The TREM 110 includes Host Interface 110(i), in which the CCU connects via a Gen2 4×PCI Express external connection to the computer 130, e.g., a compatible host computer. This provides direct memory access to the host computer 130 from the TREM 110 as well as direct memory access from the TREM 100 to the host computer 130. This bi-directional system provides configuration access to the host and DMA data transfer to the TREM 110 to minimize host processor overhead. The TREM 110 includes Aux Subsystems 110(j). For example, there are various memories and devices that function alongside the primary subsystems either in hardware or instantiated in the FPGA fabric to facilitate their operation.

The TREM 110 of the system 100 provides a modular system architecture that is capable to operate the transmitting and receiving of waveforms on any type of acoustic probe. The TREM 110 includes a flexible architecture that is able to interface with single or multiple channel probes of any size, geometry, or circuit complexity, based on the subsystems described above. Furthermore, the disclosed architecture of the system 100 provides the ability for implementations in both diagnostic applications (e.g., ultrasound imaging, Doppler range measurements, or other) and therapeutic applications (e.g., high intensity focused ultrasound (HIFU), or other).

The TREM 110 is capable of massive data transfer at real time speeds and parallel computation of received data signals. For example, the TREM 110 can simultaneously do multiple complex correlations on incoming RF data in real time (e.g., facilitated by 8 total FPGAs, one on each Tx/Rx card as well as a master FPGA on the CCU and a slave on the probe head itself, for a total of 10 in the system, as depicted in the example shown later in FIG. 2A).

The TREM 110 includes an architecture capable of modular expansion of multiple TREM units of a given or variable channels. For example, the use of FPGAs in the TREM 110 allow the system 100 to up-scale to manage the generation, transmission/reception, and processing of theoretically infinite number of discrete channels of ultrasound waveforms. Beyond the data processing capability, the TREM 110 can rapidly reconfigure itself, so it is designed to be able to rapidly switch from one configuration to the next. In an illustrative example, to produce an HD ultrasound image of the disclosed technology, a single HD image may be formed of thousands of sub-frames (e.g., such as 4000 sub-frames), each requiring a reconfiguration. The reconfiguration time needs to be minimal to minimize tissue motion. For example, the reconfiguration can include configuring (e.g., assigning) the multiplexors to address the individual elements; enabling/disabling of specific receive channels of the acoustic probe device 200, in some scenarios, if necessary/applicable; setting beamforming parameters (e.g., phases) for the AWG channels; loading waveform information data of the transmit waveform(s) for the AWG channels; setting the time gain curve for the voltage controlled attenuator; setting capture parameters, e.g., such as number of samples, for the ADC front end; and/or setting DMA addresses and transfer sizes for each receive sequence.

The TREM 110 is configured to support multiple acoustic probe designs, e.g., high impedance array designs, low impedance standard designs, and low impedance/low-frequency designs. For example, the TREM 110 can be configured to be compatible with Ultrasonix Sonix Touch HW. In some implementations, the TREM 110 is configured to produce arbitrary waveform generation for transmit on all channels. The TREM 110 allows for RF based waveform generation (e.g., RF data availability), which can be facilitated through a software endpoint.

FIG. 2A shows a block diagram of an example architecture of a 64-channel TREM 110. FIG. 2B shows an image of an exemplary transmit/receive electronics module, partially enclosed in a housing, showing various data ports for communication with the probe device 120 and the computer 130. The housing can provide an enclosure designed to a particular form factor to allow integration into desired settings, e.g., including underneath a hospital bed, on a mobile or immobile rack or shelf, or other. As shown in FIG. 2B, the system architecture allows for various Tx/Rx cards to be swapped into or out of the TREM 110 (in the housing) to allow for customizable architecture. As shown in FIG. 2A, the exemplary 64-channel TREM 110 includes eight 8-channel transmit/receive sub-modules that include a processing unit (e.g., FPGA) in communication with an 8-channel AFE connected to eight power amplifiers (e.g., for transmit waveforms) and 8-channel multiplexers. The exemplary 64-channel TREM 110 includes the control unit including a processing unit (e.g., FPGA and/or ARM processor) in communication with a master clock (e.g., clocking interface) and multiple interfaces including a PCI express interface, backplane interface, and probe interface. The exemplary 64-channel TREM 110 includes a backplane to facilitate communication of the eight 8-channel Tx/Rx sub-modules and the control unit, and to facilitate communication of an adapter (e.g., ZIF 156 adapter) and the probe device 120. For example, the ZIF 156 adapter is a passive device used as a ‘pass through’ signal adapter. In some implementations, for example, the backplane of the TREM 110 can be configured to be modular, to be directly compatible with Ultrasonix Modulo, to have full transmit/receive access to all 128 channels (e.g., 16 channels per card), to support the control unit master data and power interfaces, and/or have 55 dB+ of signal isolation. The TREM 110 includes one or more power supply units, e.g., which can include a 12V @ 750 W/−12V @ 150 W power supply and/or a 24 V @ 1200 W power supply. An example printed circuit board design featuring the backplane of TREM 110 is shown in FIG. 2C.

FIG. 2D shows a data flow diagram of the system 100 including data flow between the TREM 110, the acoustic probe device 120, and the host computer 130. The flow diagram of FIG. 2D also shows data flow connections between optional additional TREM 110 units interfaced together. As depicted in the diagram, data flow can be initiated and terminates with the controlling host processor of the computer 130. Configuration data is directly written from the host computer 130 to memories and/or devices that exist on the control unit of the TREM 110, one of the Tx/Rx units of the TREM 110, or the probe device 120. Once the configuration data is programmed, the image sequence can commence. The sequence is started and a global trigger is distributed to the Tx/Rx modules and the probe device 120 from the control unit of the TREM 110 simultaneously. For example, the Tx/Rx cards, if programmed to transmit from a specific channel, enable the required DAC Front Ends and generate the arbitrary waveform sequence. The analog signals emitted from the Tx/Rx cards travel through the backplane to the probe device 120, such that they are transduced as acoustic signals transmitted toward the volume of interest. Return acoustic signals from the volume of interest return to the probe device 120, such that they are transduced to electrical signals by the probe 120, and communicated to the TREM 110 for travel back along the same (or adjacent) analog signal paths back to the Tx/Rx cards where they are digitized by the ADC Front end. The signals from the ADC front end are streamed into a large local memory via DMA where they can then be transferred out by the control unit DMA back into allocated host memory. The system 100 has the capability of buffering multiple frames (Tx, Rx sequences) of data to allow high speed acquisition and slower host data transfer/processing. The control unit also can implement external connections suitable for slaving up to three more TREMs, for example, including clock and trigger outputs.

The control unit of the TREM 110 is configured to provide the trigger to the Tx/Rx cards, the configuration data to the Tx/Rx cards, and the waveform data to Tx/Rx cards. The control unit is configured to provide high speed IO functionality with the computer 130, e.g., to facilitate RF-based waveform data implementations. For example, the control unit of the TREM 110 is in communication with the computer 130 via a PCI Express external bus, shown in FIG. 2A. The control unit is configured to manage system configuration and sequencing (e.g., of the waveforms for transmit and receive). In some implementations, the control unit of the TREM 110 can be configured to include a high stability 100 MHz clock to Tx/Rx cards for digital-to-analog converter (DAC) and/or analog-to-digital converter (ADC) operations. In some implementations, the control unit of the TREM 110 can be configured to synchronize front-end ADC data from the Tx/Rx cards.

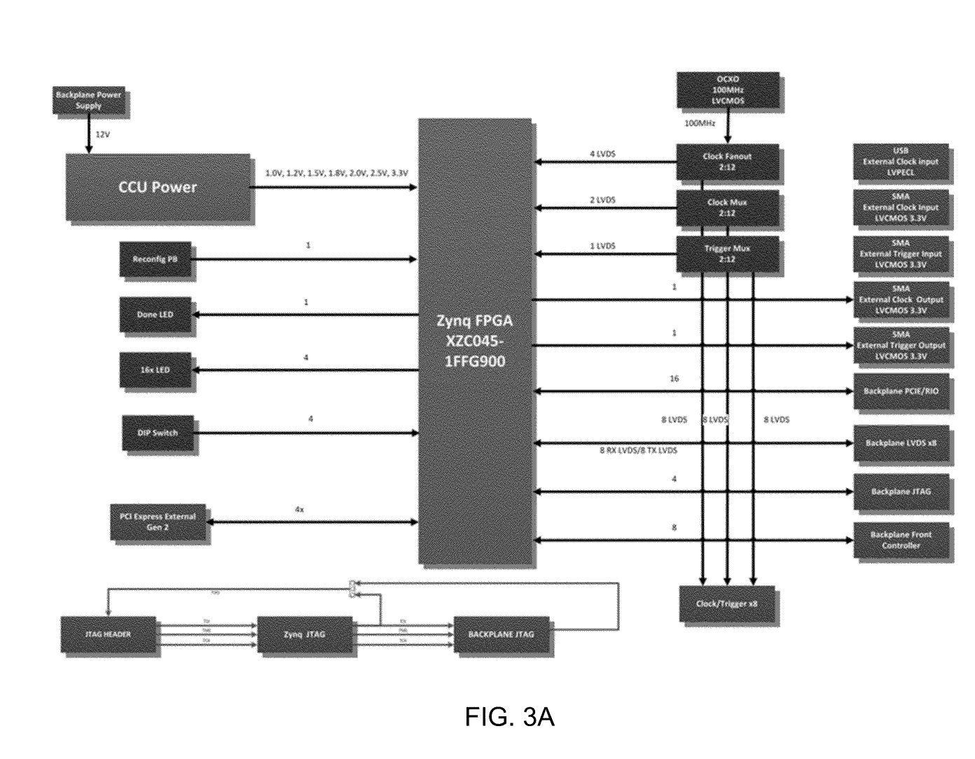

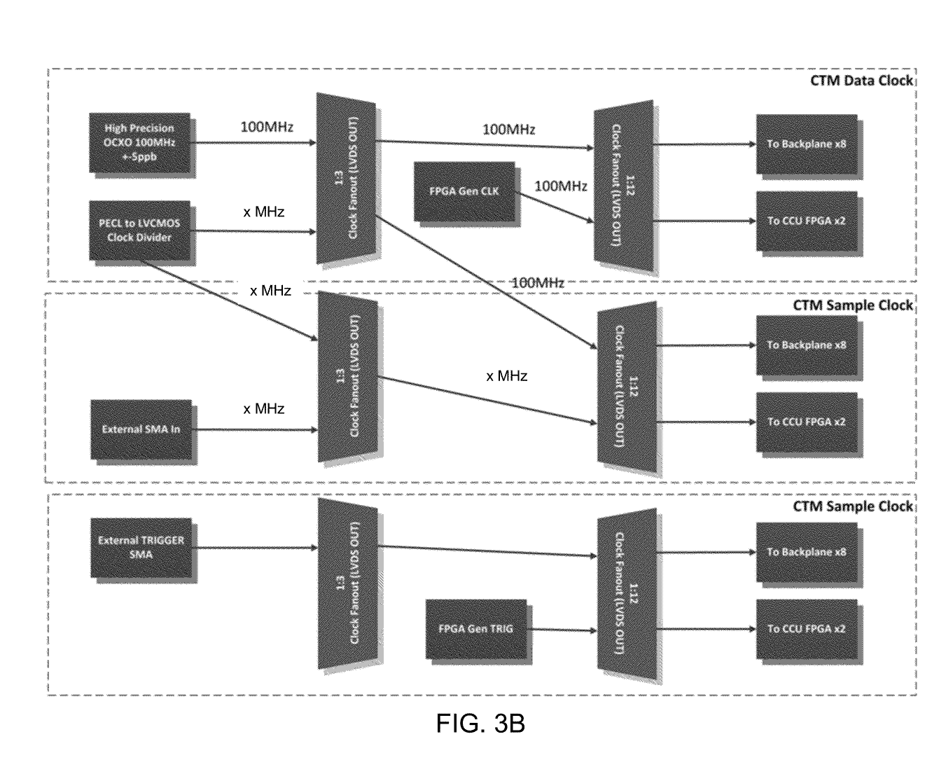

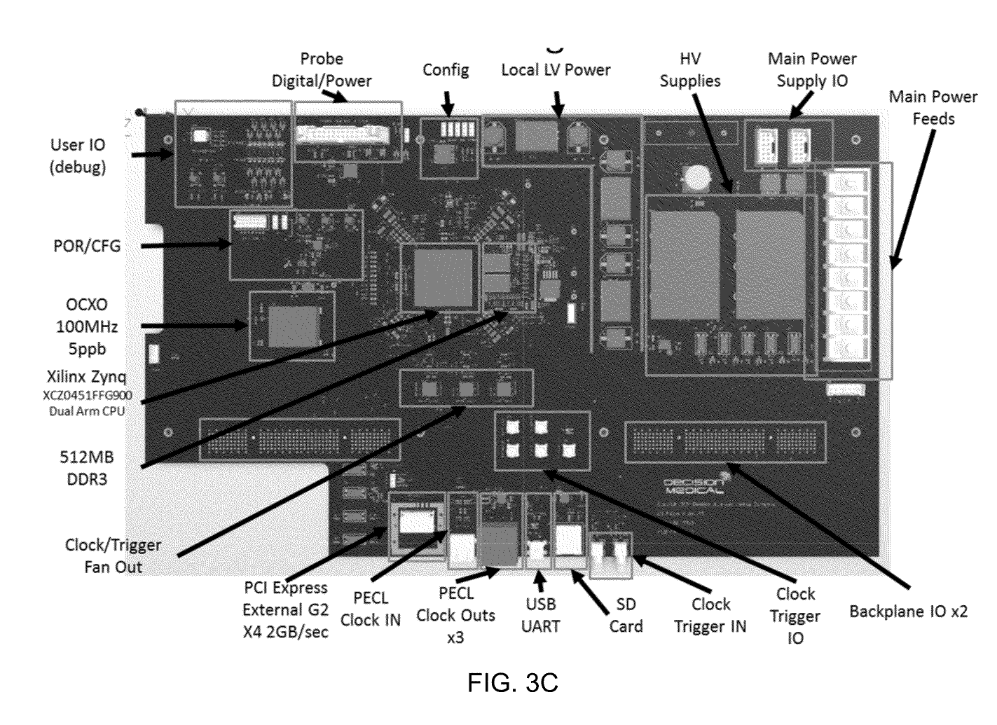

FIG. 3A shows a block diagram of an example control unit of the TREM 110 depicting device connections to and from the FPGA of the control unit. FIG. 3B shows a block diagram of clocking and triggering distribution operations, operated via the control unit, which depicts clock and trigger paths travelling from left to right. FIG. 3C shows an example printed circuit board design including the control unit of the TREM 110 depicting the arrangement and connections of various subcomponents of the control unit.

The TREM 110 can be configured to process data with the following example rates, e.g., at 50 MHz ADC/16 Bit/300 μs/15,000 samples per channel; with 24,576 receive waves per frame (e.g., 16 Tx transducers×12 segments×128 Rx transducers); and at 29.3 KB/wave. The TREM 110 can be configured to process received returned acoustic waveforms with the following example rates, e.g., at 703 MB/frame, where the Tx/Rx cards can total 4,096 MB Ram and 512 MB in CCU, where there are 5.82 frames in the buffer at any time, and with 2.275/second frame data transfer (e.g., at 2.0 GB/s).

The Tx/Rx cards of the TREM 110 are configured to provide low jitter clock division/distribution to the analog front end (AFE) and the digital-to-analog converters (DACs). The Tx/Rx cards of the TREM 110 are configured to provide power and control to the AFE and the power adapters (PAs). The Tx/Rx cards of the TREM 110 are configured to transmit arbitrary waveform sequencing, to receive data sink from the AFE and source to CCU.

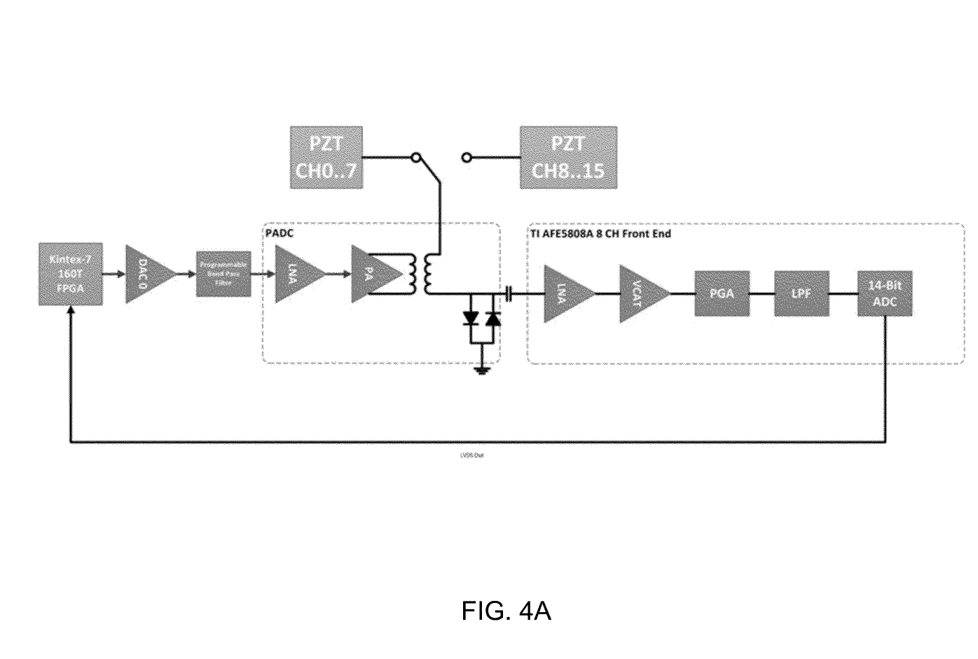

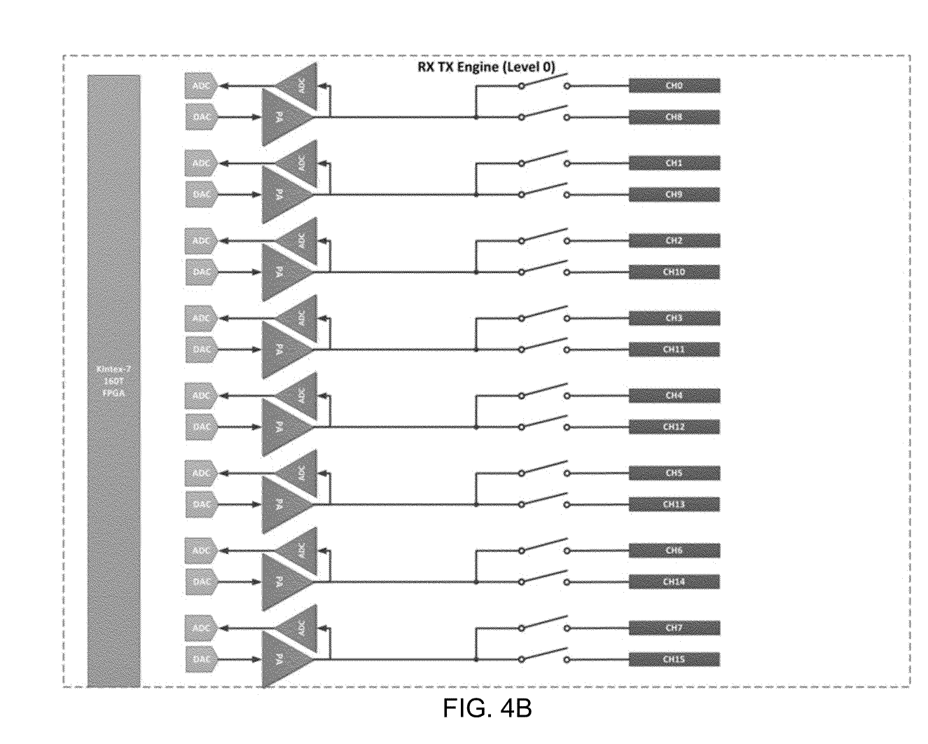

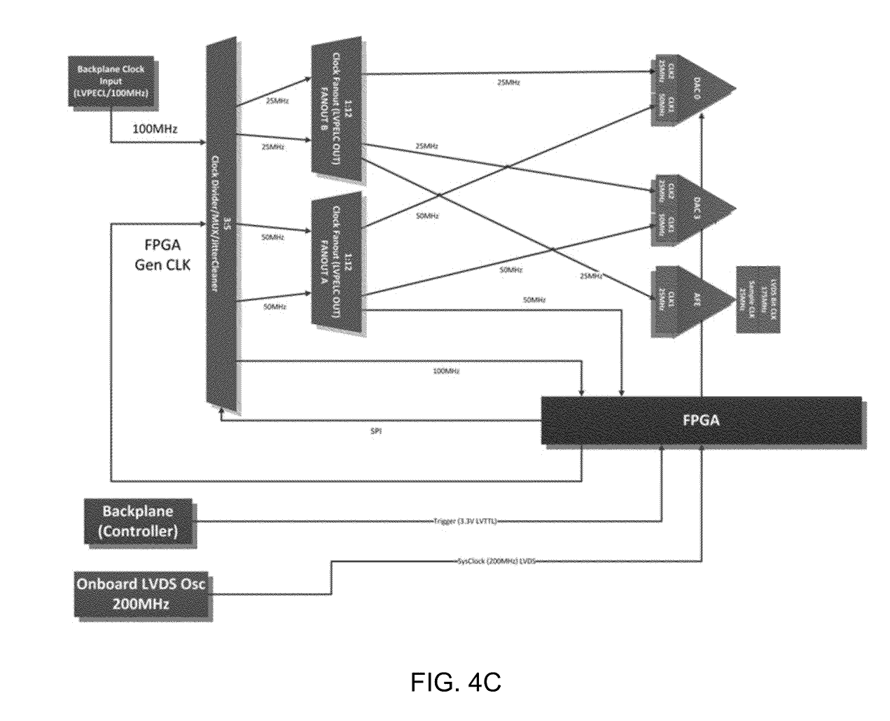

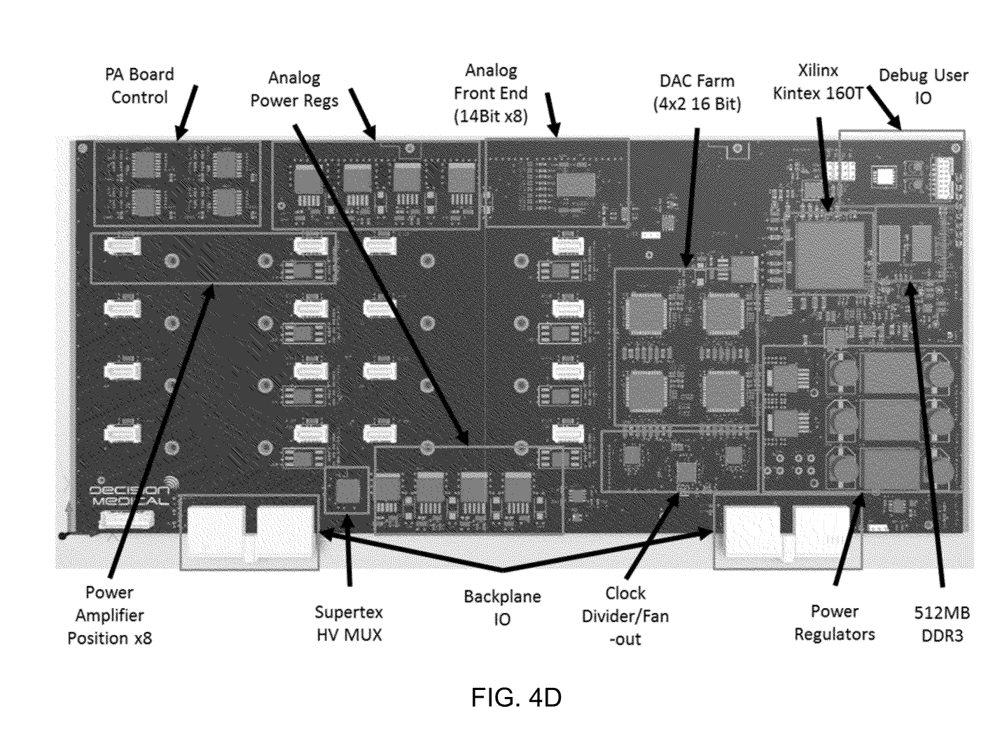

FIG. 4A shows a block diagram of an example circuit of a Tx/Rx card of the TREM 110, which shows the signal channel loop, starting and ending with the FPGA. FIG. 4B shows a block diagram of a 16-channel selection circuit of the Tx/Rx circuit. FIG. 4C shows a diagram of clock distribution operations on the Tx/Rx card. FIG. 4D shows an example printed circuit board design of a Tx/Rx card of the TREM 110.

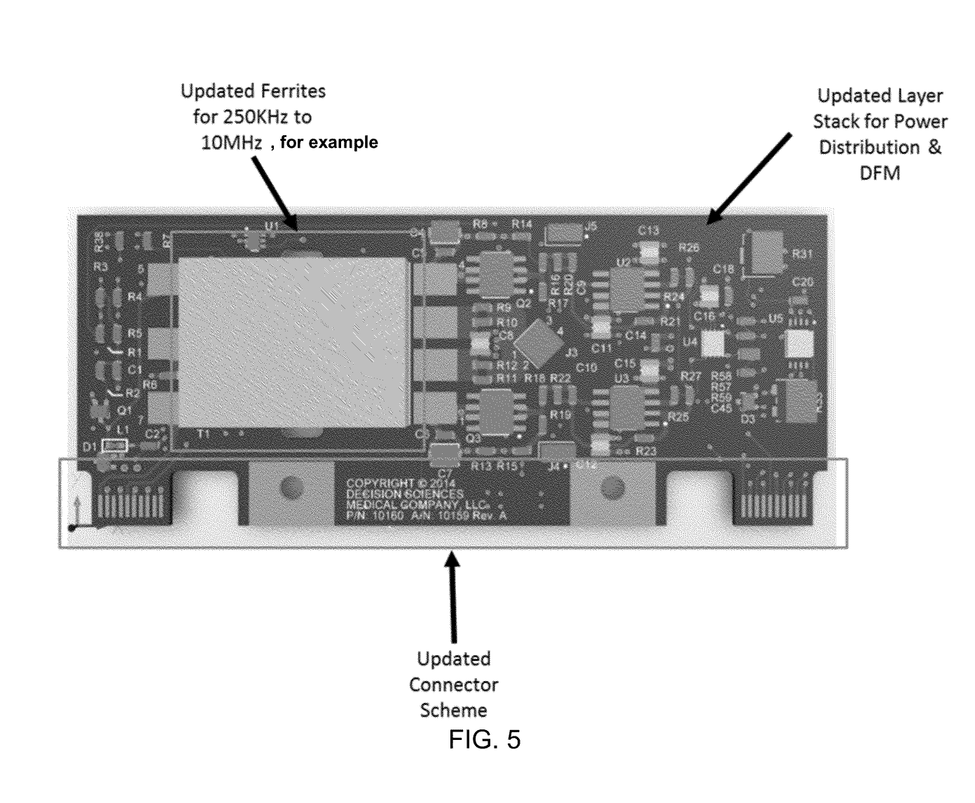

FIG. 5 shows an example printed circuit board design of a power amplifier unit of the TREM 110. The power amplifier can be configured to provide the following example features, e.g., including 80 Vₚₚ into 75Ω, 500 V/μs slew rate, PRF 7 kHz, shutdown, error signal, low distortion, 1 Vₚₚ into 50Ω input, and 250 kHz to 10 MHz bandwidth (3 dB).

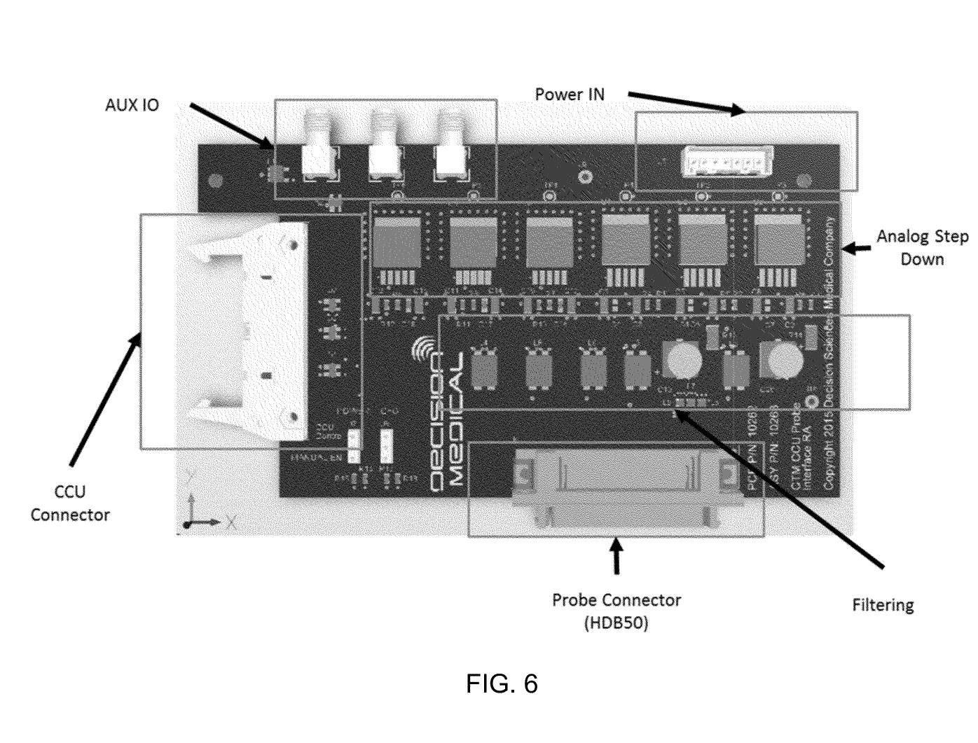

FIG. 6 shows an example printed circuit board design of the probe interface adapter unit of the TREM 110. The probe interface adapter can be configured to provide the following example features, e.g., including voltage specifications of 12 V digital (30 W), 7 V/−7 V analog (e.g., 10 W each channel), and 48 V/−48 V analog (e.g., 2 W each channel); differential digital signals (e.g., 1 in, 1 out, 1 clock); power/configuration digital reset control; and common mode filtering.

With reference back to FIG. 1, the acoustic probe device 120 can be configured to simultaneously transmit and receive the acoustic signals at the target VOI of the subject along a high density array of transducer elements arranged on one or more transducer segments based on the arbitrary or coded waveforms provided by the TREM 110. In the example shown in FIG. 1, the acoustic probe device 120 includes a probe controller unit in communication with a probe interface unit that is in communication with each probe transducer segments. For transmit, the probe controller is operable to receive the waveform information from the TREM 110 of the generated discrete waveforms carried on the multiple communication channels between the TREM 110 and the probe device 120, which are transduced by the transducer elements on the probe transducer segments. The probe interface includes a multiplexing circuit to route the waveform signals to selected transducer elements. For example, as shown on the far right side in FIG. 2D, the control path provides the probe controller configuration information directly from the control unit of the TREM 110, e.g., which can be determined by the host computer 130. In some implementations, for example, the host computer 130 can effectively pre-load the probe device 120 directly with a number of presets, and then the sequencer in the control unit can iterate through them between each transmit/receive operation. The probe device 120 can include one transducer segment or an array of multiple transducer segments arranged on a section of the housing body having a particular geometry that makes contact with a body structure of the subject during implementation of the system 100. In some embodiments, for example, the section can include a flat shape, whereas in other embodiments, the section can include a curved shape. The one or multiple transducer segments can be in communication with any or all of the channels of the TREM 110 based on the multiplexing circuitry provided on the probe controller and/or probe interface.

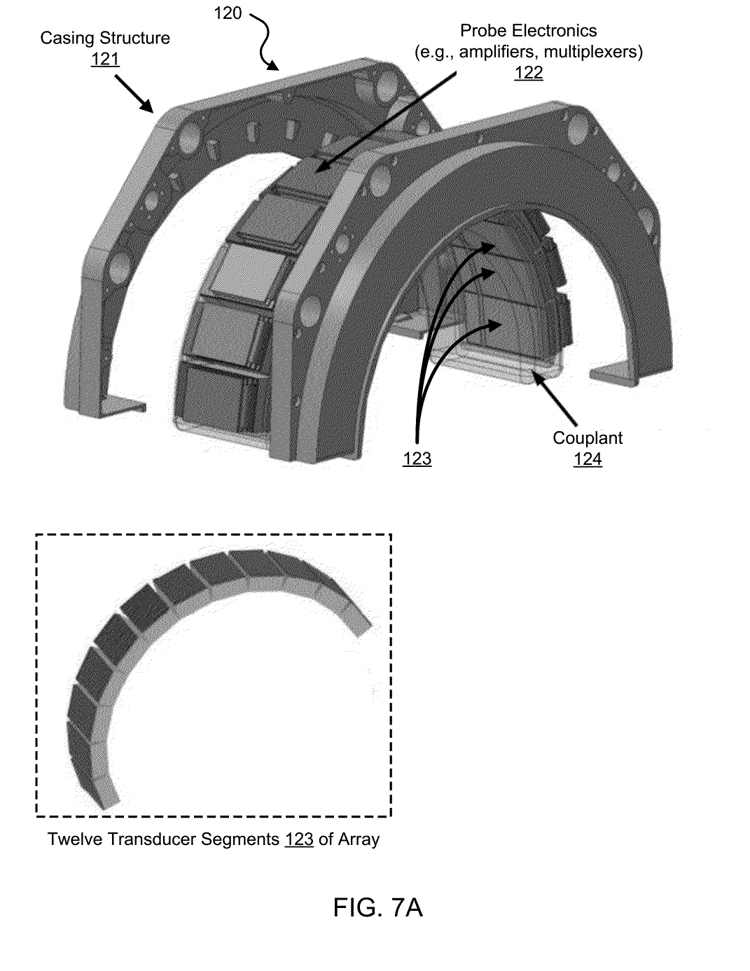

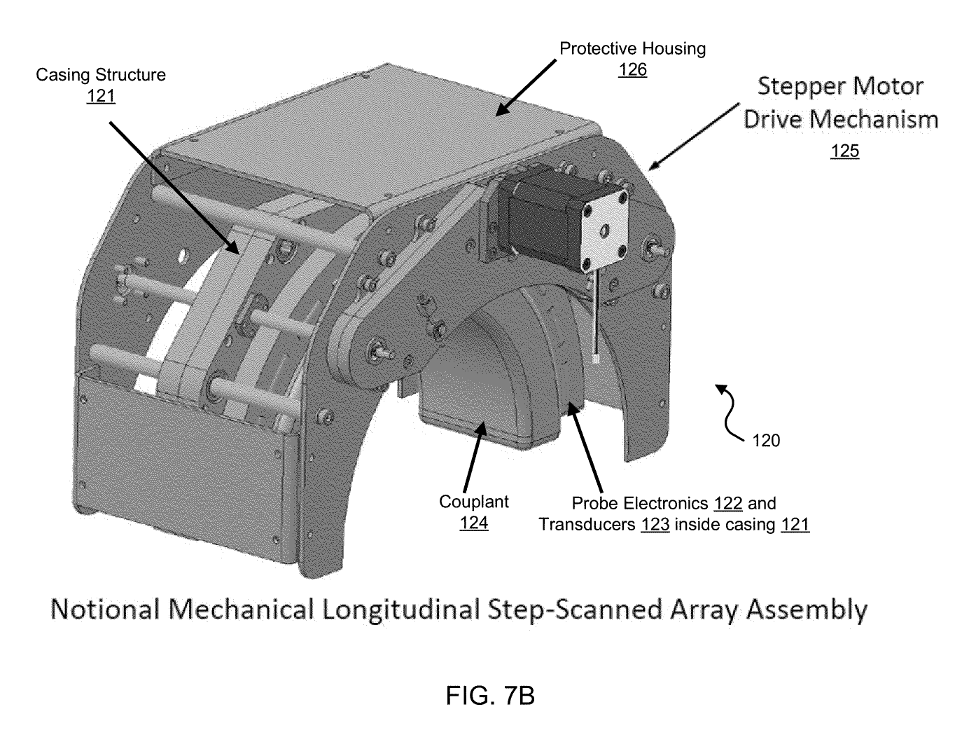

FIG. 7A shows a breakout schematic diagram of an example embodiment of the acoustic probe device 120 having a 180° curvature to present the transducer array for interfacing with a body structure of the subject where the target VOI lies. The exemplary embodiment of the acoustic probe device 120 shown in FIG. 7A includes a casing structure 121 (where at least a portion is shown), probe electronics 122 to interface with the transducers, the array of twelve transducer segments 123 arranged in a 180° curvature, where the transducer segments include an array individual transducer elements, and a couplant to acoustically couple the transducers 123 with a receiving medium of the target VOL In other implementations, for example, the acoustic probe device 120 includes a 360° probe, which can include 24 transducer segments arranged along a 360° curvature of the probe device 120. Additional information pertaining to the acoustic probe device 120 of the disclosed synthetic aperture ultrasound system technology, e.g., including exemplary embodiments of acoustic signal transmission couplant devices of the disclosed systems, is described in the attached document of U.S. Provisional Patent Application No. 62/120,839, titled ACOUSTIC SIGNAL TRANSMISSION COUPLANTS, which is included as part of this disclosure in this patent document.

FIG. 7B shows a schematic illustration of an exemplary embodiment of the acoustic probe device 120 that includes a stepper motor drive unit to 125 controllably move components of the probe device 120, e.g., including the transducer segments 123, with respect to the receiving medium of the target VOI to which the probe device 120 is interfaced. In some implementations, for example, the acoustic probe device 120 can include a protective housing 126 that at least partially encloses the casing structure 121, probe electronics 122, and array of transducer segments 123. In other exemplary embodiments, the transmitting and receiving positions of the transducers of the probe device 120 can be moved by physically moving the probe device 120 and tracking the motion of the probe device 120 using an inertial motion unit (IMU).

In some embodiments of the system 110, the system 110 includes four TREM 100 unit working together, each of which can support up to 12 transducer segments in various configurations, e.g., including 180° half-rings, 360° rings, and more complex surfaces such as a superset of the 180° half-rings (e.g., such as four curved ring components that make a half pipe for scanning large areas like legs, torsos, head, etc.). The disclosed system architecture can incorporate more TREM 110 units into a single massive scanner.

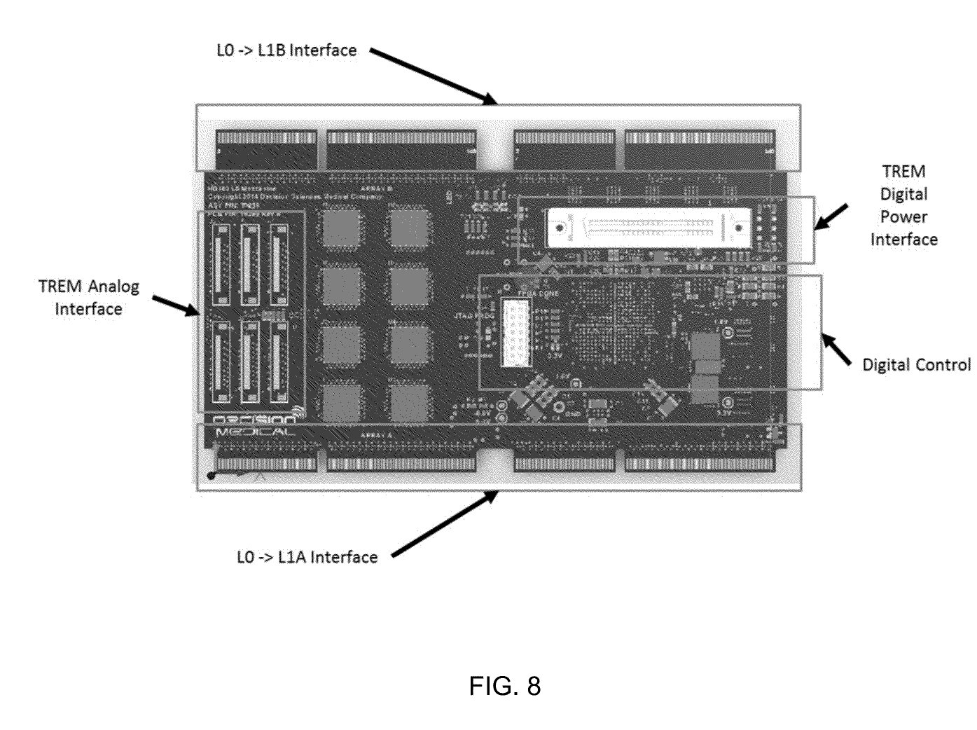

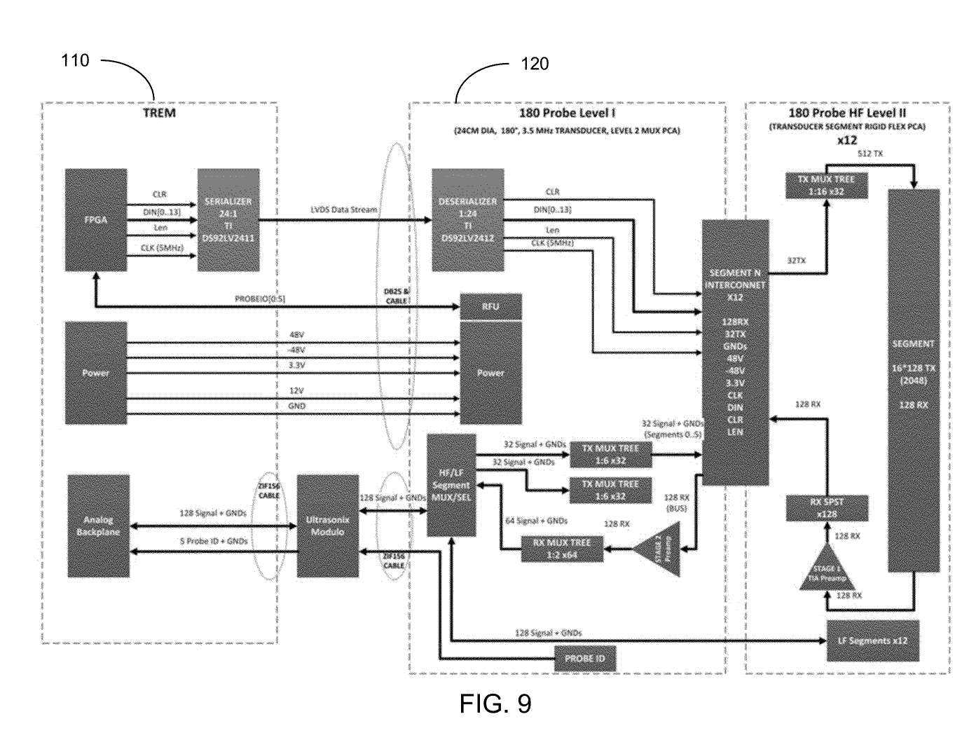

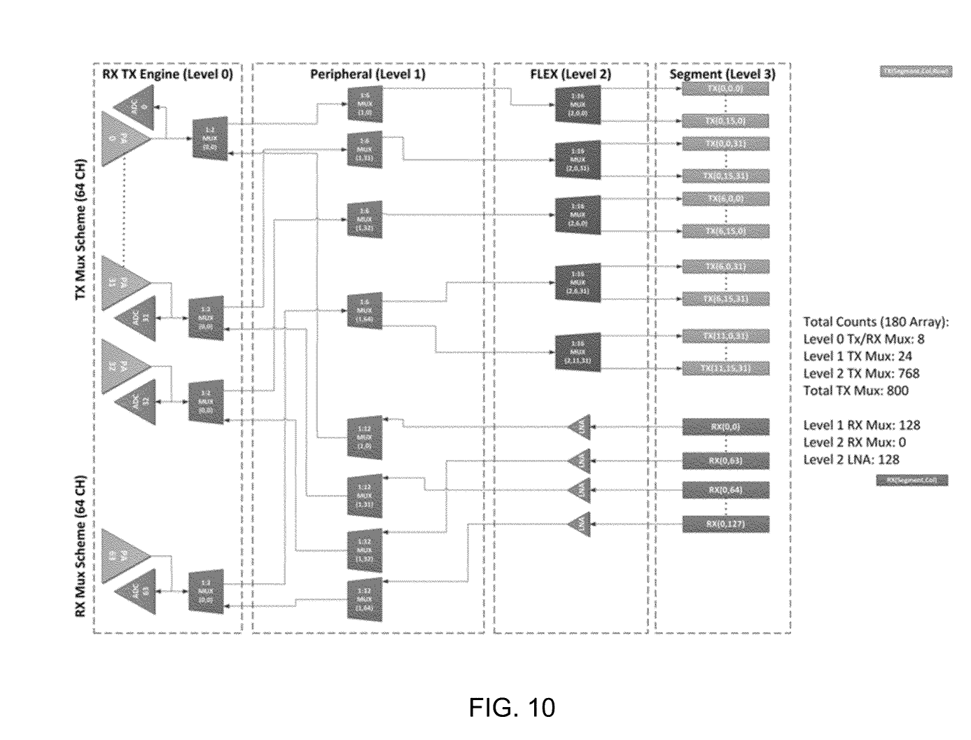

FIG. 8 shows an example printed circuit board design for the transducer array L0 probe unit of the acoustic probe device 120, which is in communication with the adapter of the TREM 110. FIG. 9 shows an example data flow diagram for probe data between the TREM 110 and the probe device 120, depicting a signal level interconnection configuration between the TREM, Probe Controller, and array PCAs. FIG. 10 shows a block diagram of the multiplexer units in the probe device 120.

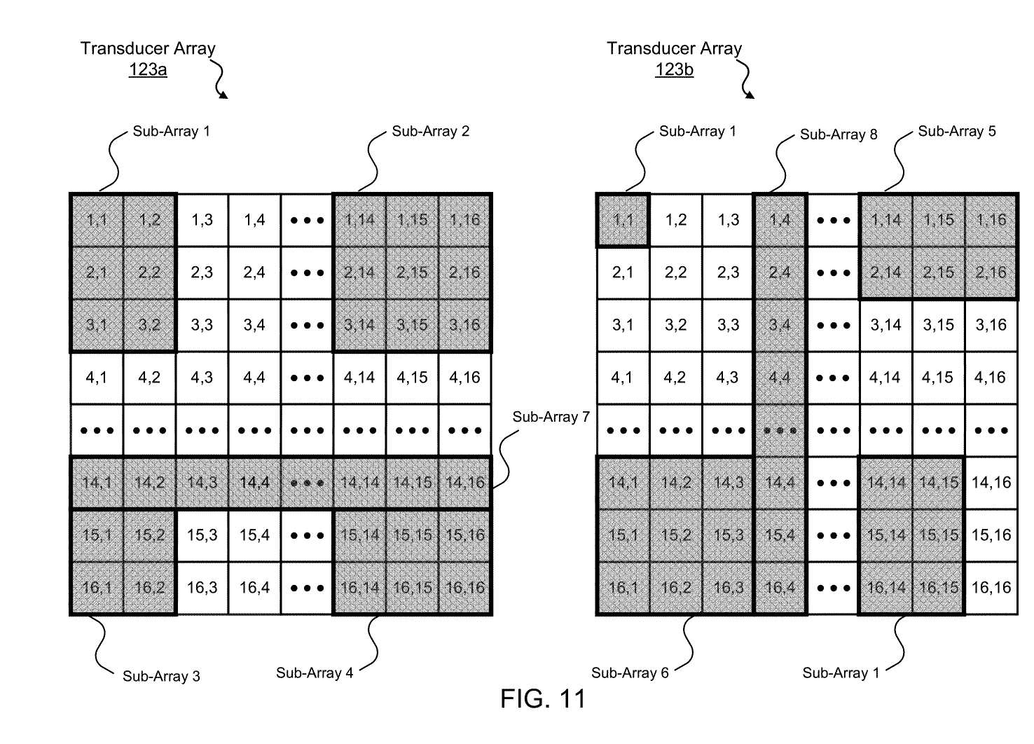

In some implementations, for example, various arrays on one or more transducer segments 123 can be selected for transmitting acoustic waveforms and receiving returned acoustic waveforms. Such selected arrays can include various combinations of transducer elements in one or more sub-arrays across, which can be selected on one or multiple transducer segments 123, to generate the arbitrary waveforms or the orthogonal coded acoustic waveforms. FIG. 11 shows a diagram of an exemplary selected transducer array including selected sub-arrays of particular transducer elements on multiple transducer segments 123a and 123b for generating a composite ultrasound beam of the disclosed technology. For example, a sub-array can include combinations of individual transducer elements on one transducer segment or among a plurality of the transducer segments, as shown in FIG. 11. In the example of FIG. 11, the selected transducer array includes 79 individual transducer elements arranged in eight sub-arrays of the multiple transducer element 123a and 123b. In this example, the 79 individual transducer elements are selected to transmit (e.g., either sequentially, simultaneously or randomly) the individual waveforms or aspects of a composite waveform, e.g., in which the individual waveforms can include individual arbitrary waveforms or individual orthogonal, coded acoustic waveforms. For example, a sub-array 1 includes nine transducer elements on the transducer segment 123a at (row, col): 1,1; 1,2; 2,1; 2,2; 3,1; 3,2; and on the transducer segment 123b at (row, col): 1,1; 14,14; 14,15; 15,14; 15,15; 16,14; 16,15. A sub-array 2 includes transducer elements on the transducer segment 123a at (row, col): 1,14; 1,15; 1,16; 2,14; 2,15; 2,16; 3,14; 3,15; 3,16. A sub-array 3 includes transducer elements on the transducer segment 123a at (row, col): 15,1; 15,2; 16,1; 16,2. A sub-array 4 includes transducer elements on the transducer segment 123a at (row, col): 15,14; 15,15; 15,16; 16,14; 16,15; 16,16. A sub-array 5 includes transducer elements on the transducer segment 123b at (row, col): 1,14; 1,15; 1,16; 2,14; 2,15; 2,16. A sub-array 6 includes transducer elements on the transducer segment 123b at (row, col): 14,1; 14,2; 14,3; 15,1; 15,2; 15,3; 16,1; 16,2; 16,3. A sub-array 7 includes transducer elements on the transducer segment 123a at (row, col): 14,1; 14,2; 14,3; . . . 14:16. A sub-array 8 includes transducer elements on the transducer segment 123b at (row, col): 1,4; 2,4; 3,4; . . . 16,4. Configurations of the sub-arrays can be produced using a switching element (e.g., such as a multiplexer unit) interfaced between waveform generators and the transducer array, as shown FIG. 1.

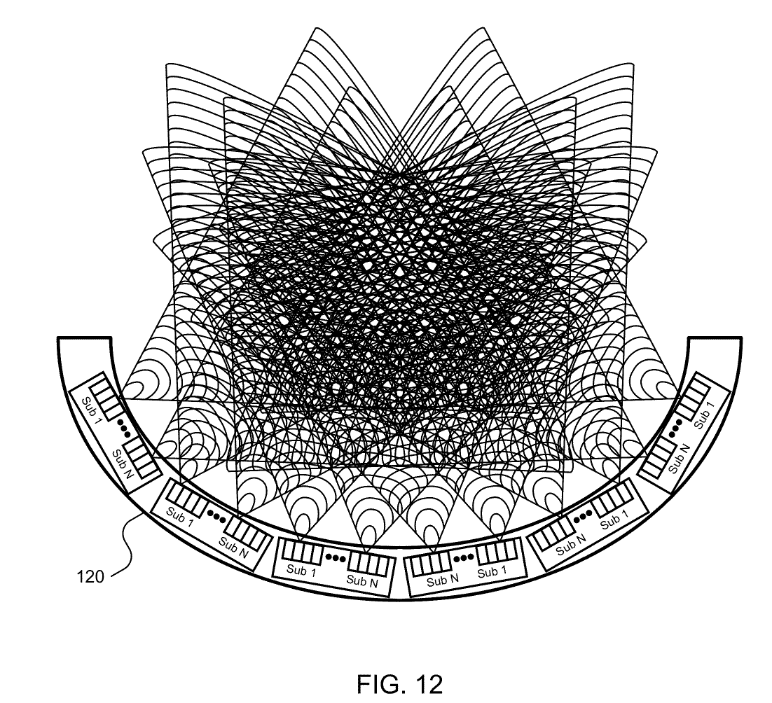

FIG. 12 shows a diagram of exemplary composite ultrasound beams generated by transducer sub-arrays on multiple transducer segments that forms a synthetic aperture beam from multiple transmitting positions along a 180° curvature of the acoustic probe 120. As shown in the diagram, an acoustic probe 120 includes multiple transducer segments 123 used to form one or more real aperture sub-arrays Sub 1, Sub 2, . . . Sub N on one or more of the transducer segments 123. Some or all of the transducer elements that form the transducer array can transmit (e.g., either sequentially, simultaneously or randomly) one or more composite acoustic waveforms of individual, mutually orthogonal, coded acoustic waveforms transmitted to a target VOI from multiple sub-array phase center positions to form a synthetic aperture for ultrasound imaging. In some implementations, different transducer elements on the transducer segments 123 can be selected to form the receive array to receive the returned acoustic waveforms corresponding to the transmitted acoustic waveform (formed based on the individual, mutually orthogonal, coded acoustic waveforms), in which the received acoustic waveforms are scattered back and returned (e.g., reflected, refracted, diffracted, delayed, and/or attenuated) from at least part of the VOL Whereas, in some implementations, some or all of the transducer elements that form the transmit array can also receive the returned acoustic waveforms corresponding to the transmitted acoustic waveform. The received individual acoustic waveforms thereby form one or more received composite waveforms that correspond to the transmitted composite acoustic waveforms. The composite acoustic waveform can be generated based on a composite synthetic waveform formed of multiple spread-spectrum, wide instantaneous bandwidth, coded waveforms used to generate the individual acoustic waveforms. The individual, composite, acoustic waveforms can be transducted by one or more of the sub-arrays of the transducer array. The transducer array can be positioned at multiple physical positions along a known path, and/or multiple beam-steering positions, such that the phase center is positioned mechanically, electronically or both mechanically and electronically in the successive positions, e.g., forming a synthetic aperture.

### EXAMPLES

The following examples are illustrative of several embodiments of the present technology. Other exemplary embodiments of the present technology may be presented prior to the following listed examples, or after the following listed examples.

In one example of the present technology (example 1), a synthetic aperture acoustic waveform system includes (i) a waveform generation and processing device, and (ii) an acoustic probe device. The (i) waveform generation and processing device includes a waveform generator in communication with one or more waveform synthesizers to generate one or more waveforms according to waveform information provided by the waveform generator, and a controller unit including a memory to store data and a processing unit coupled to the memory to process data. The (ii) acoustic probe device includes a housing body including a shaped section to interface a body structure of a biological subject, one or more transducer segments including an array of transducer elements arranged on the shaped section of the housing body to transmit acoustic waveforms corresponding to the one or more waveforms generated by the waveform generation and processing device toward a target volume in the biological subject and to receive returned acoustic waveforms that return from at least part of the target volume, an acoustic coupling component to conduct the acoustic waveforms between the transducer elements and the body structure of the biological subject when in contact with the acoustic coupling component, and a multiplexing unit in communication with the controller unit and the array of transducer elements to select one or more transducing elements of the array to transduce the waveforms into the corresponding acoustic waveforms, and to select one or more transducing elements of the array to receive the returned acoustic waveforms. The (i) waveform generation and processing device includes: an array of analog to digital (A/D) converters to convert the received returned acoustic waveforms received by the array of transducer elements of the acoustic probe device from analog format to digital format as a received waveform that includes information of at least part of the target volume, one or more amplifiers in communication with the one or more waveform synthesizers to modify the individual orthogonal coded waveforms provided to the acoustic probe device for transmission, and one or more pre-amplifiers in communication with the acoustic probe device and the array of A/D converters to modify the received returned acoustic waveforms provided to the A/D converters.

Example 2 includes the system of example 1, in which the processing unit of the controller unit is operable to process the received returned acoustic waveforms to produce a data set including the information of at least part of the target volume.

Example 3 includes the system of example 1, in which the stored data includes the digital format of the received returned acoustic waveforms, the corresponding synthesized waveforms, and corresponding position data of transducer elements operated to transmit and transducer elements operated to receive in transmitting and receiving positions, respectively.

Example 4 includes the system of example 1, in which the processing unit includes a digital signal processor.

Example 5 includes the system of example 1, in which the waveform generation and processing device includes a master clock in communication with the controller unit to synchronize time in at least one of the elements of the system.

Example 6 includes the system of example 1, in which the waveform generation and processing device is in communication with a computer including a processor and a memory, such that the controller unit of the waveform generation and processing device is configured to transfer processed data including the information of at least part of the target volume to the computer.

Example 7 includes the system of example 6, in which the computer is configured to produce an image of at least part of the target volume based on the information, in which the computer includes a visual display to display the image, and a user input terminal to receive user input data including a mode of operation for operation of the system.

Example 8 includes the system of example 1, in which the processing unit of the controller unit is operable to process the received returned acoustic waveforms to produce a data set including information of at least part of the target volume that includes range data and associated range rate data, e.g., which may be referred to as Doppler frequency shift data, from at least part of the target.

Example 9 includes the system of example 1, in which transducer elements selected to transmit the acoustic waveforms are operable for moving in one dimension, two dimensions, or three dimensions to one or more transmit positions to transmit the acoustic waveforms.

Example 10 includes the system of example 1, in which transducer elements selected to receive the returned acoustic waveforms are operable for moving in one dimension, two dimensions, or three dimensions to one or more receive positions to receive the returned acoustic waveforms.

Example 11 includes the system of example 1, in which transducer elements selected to transmit the acoustic waveforms and to receive the returned acoustic waveforms are operable for moving in one dimension, two dimensions, or three dimensions to one or more transmit positions to transmit the acoustic waveforms and to one or more receive positions to receive the returned acoustic waveforms, respectively.

Example 12 includes the system of example 1, in which the transducer elements are capable of moving separately in the one dimension, two dimensions, or three dimensions from the other transducer segments.

Example 13 includes the system of example 1, in which the number of transducer elements selected to transmit the acoustic waveforms is greater than the number of transducer elements selected to transmit the returned acoustic waveforms.

Example 14 includes the system of example 1, in which the number of transducer elements selected to receive the returned acoustic waveforms is greater than the number of transducer elements selected to transmit the acoustic waveforms.

Example 15 includes the system of example 1, in which the biological subject includes a human or non-human animal.

Example 16 includes the system of example 1, in which the target volume includes a tissue structure of the biological subject, and the shaped section of the probe device is in contact with the body structure of the biological subject.

Example 17 includes the system of example 16, in which the body structure includes an abdomen, a thorax, a neck including the throat, an arm, a leg, a knee joint, a hip joint, an ankle joint, an elbow joint, a shoulder joint, a wrist joint, a breast, a genital, or a head including the cranium.

Example 18 includes the system of example 16, in which the shaped section includes a curved section of the housing body, the curved section having a curvature to facilitate complete contact with the body structure, such that the acoustic coupling component is in direct contact with skin of the body structure.

Example 19 includes the system of example 16, in which the biological structure includes a cancerous or noncancerous tumor, an internal legion, a connective tissue sprain, a tissue tear, or a bone.

Example 20 includes the system of example 1, in which the waveform generation and processing device is operable to generate any arbitrary waveforms that can be described mathematically.

Example 21 includes the system of example 20, in which the waveform generation and processing device is operable to beamform and steer the arbitrary waveforms.

Example 22 includes the system of example 20, in which the arbitrary waveforms include one or more of rectangular pulses, triangular pulses, Gaussian pulses, sinusoidal pulses, sinc pulse, Mexican hat wavelet pulses, Haar wavelet pulses, linear FM chirped pulses, hyperbolic FM chirped pulses, or combinations thereof.

Example 23 includes the system of example 1, in which the waveform generation and processing device is operable to generate a composite waveform including two or more of individual orthogonal coded waveforms corresponding to different frequency bands that are generated by the one or more waveform synthesizers according to the waveform information provided by the waveform generator, in which the individual orthogonal coded waveforms are mutually orthogonal to each other and correspond to different frequency bands, such that each of the individual orthogonal coded waveforms includes a unique frequency with a corresponding phase.

Example 24 includes the system of example 23, in which each of the individual orthogonal coded waveforms includes a plurality of amplitudes and a plurality of phases that are individually amplitude weighted and individually phase weighted, respectively.

Example 25 includes the system of example 23, in which the waveform generation and processing device is operable to determine a frequency band, an amplitude, a time-bandwidth product parameter, and a phase parameter of each individual orthogonal coded waveform.

Example 26 includes the system of example 25, in which the phase parameter is determined from a set of a pseudo-random numbers or from a set of deterministic numbers.

Example 27 includes the system of example 23, in which the individual orthogonal coded waveforms include coherent waveforms.

Example 28 includes the system of example 23, further including a second acoustic probe device in communication with the waveform generation and processing device to transmit the acoustic waveforms and receive the returned acoustic waveforms.

Example 29 includes the system of example 23, further including a second waveform generation and processing device in communication with the waveform generation and processing device to provide additional channels to provide the one or more waveforms to the acoustic probe device or multiple acoustic probe devices.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A synthetic aperture acoustic waveform system, comprising:
(i) a waveform generation and processing device, including:
a waveform generator in communication with one or more waveform synthesizers to generate one or more waveforms according to waveform information provided by the waveform generator, and
a controller unit including a memory to store data and a processing unit coupled to the memory to process data; and
(ii) an acoustic probe device, including:
a housing body including a shaped section to interface a body structure of a biological subject,
one or more transducer segments comprising an array of transducer elements arranged on the shaped section of the housing body to transmit acoustic waveforms corresponding to the one or more waveforms generated by the waveform generation and processing device toward a target volume in the biological subject and to receive returned acoustic waveforms that return from at least part of the target volume, and
an acoustic coupling component to conduct the acoustic waveforms between the transducer elements and the body structure of the biological subject when in contact with the acoustic coupling component, and
a multiplexing unit in communication with the controller unit and the array of transducer elements to select one or more transducing elements of the array to transduce the waveforms into the corresponding acoustic waveforms, and to select one or more transducing elements of the array to receive the returned acoustic waveforms;
wherein: the waveform generation and processing device includes:
an array of analog to digital (A/D) converters to convert the received returned acoustic waveforms received by the array of transducer elements of the acoustic probe device from analog format to digital format as a received waveform that includes information of at least part of the target volume,
one or more amplifiers in communication with the one or more waveform synthesizers to modify the individual orthogonal coded waveforms provided to the acoustic probe device for transmission, and
one or more pre-amplifiers in communication with the acoustic probe device and the array of A/D converters to modify the received returned acoustic waveforms provided to the A/D converters.

2. The system of claim 1, wherein the processing unit of the controller unit is operable to process the received returned acoustic waveforms to produce a data set including the information of at least part of the target volume.

3. The system of claim 1, wherein the stored data includes the digital format of the received returned acoustic waveforms, the corresponding synthesized waveforms, and corresponding position data of transducer elements operated to transmit and transducer elements operated to receive in transmitting and receiving positions, respectively.

4. The system of claim 1, wherein the processing unit comprises a digital signal processor.

5. The system of claim 1, wherein the waveform generation and processing device includes a master clock in communication with the controller unit to synchronize time in at least one of the elements of the system.

6. The system of claim 1, wherein the waveform generation and processing device is in communication with a computer including a processor and a memory, such that the controller unit of the waveform generation and processing device is configured to transfer processed data including the information of at least part of the target volume to the computer.

7. The system of claim 6, wherein the computer is configured to produce an image of at least part of the target volume based on the information, wherein the computer includes a visual display to display the image, and a user input terminal to receive user input data including a mode of operation for operation of the system.

8. The system of claim 1, wherein the processing unit of the controller unit is operable to process the received returned acoustic waveforms to produce a data set including information of at least part of the target volume that includes range data and associated range rate data from at least part of the target.

9. The system of claim 1, wherein transducer elements selected to transmit the acoustic waveforms are operable for moving in one dimension, two dimensions, or three dimensions to one or more transmit positions to transmit the acoustic waveforms.

10. The system of claim 1, wherein transducer elements selected to receive the returned acoustic waveforms are operable for moving in one dimension, two dimensions, or three dimensions to one or more receive positions to receive the returned acoustic waveforms.

11. The system of claim 1, wherein transducer elements selected to transmit the acoustic waveforms and to receive the returned acoustic waveforms are operable for moving in one dimension, two dimensions, or three dimensions to one or more transmit positions to transmit the acoustic waveforms and to one or more receive positions to receive the returned acoustic waveforms, respectively.

12. The system of claim 1, wherein the transducer elements are capable of moving separately in the one dimension, two dimensions, or three dimensions from the other transducer segments.

13. The system of claim 1, wherein the number of transducer elements selected to transmit the acoustic waveforms is greater than the number of transducer elements selected to transmit the returned acoustic waveforms.

14. The system of claim 1, wherein the number of transducer elements selected to receive the returned acoustic waveforms is greater than the number of transducer elements selected to transmit the acoustic waveforms.

15. The system of claim 1, wherein the biological subject includes a human or non-human animal.

16. The system of claim 1, wherein the target volume includes a tissue structure of the biological subject, and the shaped section of the probe device is in contact with the body structure of the biological subject.

17. The system of claim 16, wherein the body structure includes an abdomen, a thorax, a neck including the throat, an arm, a leg, a knee joint, a hip joint, an ankle joint, an elbow joint, a shoulder joint, a wrist joint, a breast, a genital, or a head including the cranium.

18. The system of claim 16, wherein the shaped section includes a curved section of the housing body, the curved section having a curvature to facilitate complete contact with the body structure, such that the acoustic coupling component is in direct contact with skin of the body structure.

19. The system of claim 16, wherein the biological structure includes a cancerous or noncancerous tumor, an internal legion, a connective tissue sprain, a tissue tear, or a bone.

20. The system of claim 1, wherein the waveform generation and processing device is operable to generate an arbitrary waveform that is characterized based on a mathematical relationship.

21. The system of claim 20, wherein the waveform generation and processing device is operable to beamform and steer the arbitrary waveforms.

22. The system of claim 20, wherein the arbitrary waveform includes one or more of rectangular pulses, triangular pulses, Gaussian pulses, sinusoidal pulses, sinc pulse, Mexican hat wavelet pulses, Haar wavelet pulses, linear FM chirped pulses, hyperbolic FM chirped pulses, or combinations thereof.

23. The system of claim 1, wherein the waveform generation and processing device is operable to generate a composite waveform comprising two or more of individual orthogonal coded waveforms corresponding to different frequency bands that are generated by the one or more waveform synthesizers according to the waveform information provided by the waveform generator, wherein the individual orthogonal coded waveforms are mutually orthogonal to each other and correspond to different frequency bands, such that each of the individual orthogonal coded waveforms includes a unique frequency with a corresponding phase.

24. The system of claim 23, wherein each of the individual orthogonal coded waveforms includes a plurality of amplitudes and a plurality of phases that are individually amplitude weighted and individually phase weighted, respectively.

25. The system of claim 23, wherein the waveform generation and processing device is operable to determine a frequency band, an amplitude, a time-bandwidth product parameter, and a phase parameter of each individual orthogonal coded waveform.

26. The system of claim 25, wherein the phase parameter is determined from a set of a pseudo-random numbers or from a set of deterministic numbers.

27. The system of claim 23, wherein the individual orthogonal coded waveforms include coherent waveforms.

28. The system of claim 23, further comprising:
a second acoustic probe device in communication with the waveform generation and processing device to transmit the acoustic waveforms and receive the returned acoustic waveforms.

29. The system of claim 23, further comprising:
a second waveform generation and processing device in communication with the waveform generation and processing device to provide additional channels to provide the one or more waveforms to the acoustic probe device or multiple acoustic probe devices.
